(19) United States
(12) Patent Application Publication
(10) Pub. No.: US 2010/0137971 A1
(43) Pub. Date: Jun. 3, 2010

(54) Stent with a Structure of a Biocorrodible Material and a Controlled Corrosion Behavior

(75) Inventors: Daniel Lootz, Rostock (DE); Bernd Block, Rostock (DE)

Correspondence Address: BIOTECH BEACH LAW GROUP , PC, 5677 OBERLIN DRIVE, SUITE 204, SAN DIEGO, CA, 92121, US

(73) Assignee: BIOTRONIK VI PATENT AG, Baar (CH)

(21) Appl. No.: 12/576,025

(22) Filed: Oct. 8, 2009

(30) Foreign Application Priority Data
Dec. 1, 2008 (DE) 10 2008 044 221.6

(51) Int. Cl. A61F 2/06; B23K 26/00; B23K 15/00

(52) U.S. Cl. 623/1.15; 219/121.85; 219/121.35

(57) ABSTRACT

The invention relates to a stent with a structure of a biocorrodible magnesium alloy or tungsten alloy, which comprises a multiplicity of web sections connected to one another, wherein (i) the structure has a support structure of a number of first web sections connected to one another, which are designed to perform a function supporting the vascular wall or preserving the mechanical integrity of the stent after an expansion of the stent for a predetermined period of time and (ii) at least one second web section is present, which a) is directly connected to a selected first web section, b) does not perform a function supporting the vascular wall or preserving the mechanical integrity of the stent after the expansion of the stent for the predetermined period of time, and c) has a smaller average grain size than the first web sections of the support structure from (i).

### CROSS REFERENCE TO RELATED APPLICATIONS

This invention claims benefit of priority to Germany patent application serial number DE 10 2008 044 221.6, filed on Dec. 1, 2008; the contents of which are herein incorporated by reference in their entirety.

### TECHNICAL FIELD

The invention relates to a stent with a structure of a biocorrodible metallic material and a controlled corrosion behavior, as well as a method for the production of a stent of this type.

### BACKGROUND OF THE INVENTION

The implantation of stents has established itself as one of the most effective therapeutic measures in the treatment of vascular diseases. Stents have the purpose of assuming a support function in the hollow organs of a patient. Stents of typical construction have a filigree support structure made of metallic struts for this purpose, which structure is first provided in a compressed form for introduction into the body and is expanded at the location of application. One of the main areas of application of stents of this type is permanently or temporarily expanding and keeping open vascular constrictions, in particular constrictions (stenoses) of the coronary vessels. In addition, for example, aneurysm stents are also known, which are used to support damaged vascular walls.

Stents have a tubular body through which the blood flow continues unobstructed, and the circumferential wall of which performs a support function for the vascular wall. The body is often formed as a latticed structure, with a multiplicity of individual web sections connected to one another. Furthermore, the design specification for the latticed structure must allow the stent to be inserted in a compressed state having a small external diameter up to the constricted point of the particular vessel to be treated and to be expanded there with the aid of a balloon catheter, for example, enough that the vessel has the desired, enlarged internal diameter. In order to avoid unnecessary vascular damage, the stent should recoil elastically at most slightly or not at all after the expansion and removal of the balloon, so that the stent has to be expanded only slightly beyond the desired final diameter during expansion. Further structural requirements are a uniform area coverage and a structure that allows a certain flexibility with respect to the longitudinal axis of the stent. Structurally, the individual web sections forming a latticed structure can be divided into those with a support function for the vascular wall and bearing function (i.e., the function ensuring the mechanical integrity of the implant) and those without involvement in the cited structural functions. The mesh of the former web sections is referred to below as the bearing structure. In practice, the stent is typically molded from a metallic material to implement the cited structural properties.

In addition to the mechanical properties of a stent, the stent should comprise a biocompatible material in order to avoid rejection reactions. Currently, stents are used in approximately 70% of all percutaneous interventions; however, an in-stent restenosis occurs in 25% of all cases because of excess neointimal growth, which is caused by a strong proliferation of the arterial smooth muscle cells and a chronic inflammation reaction. Various solution approaches are used to reduce the restenosis rates.

One approach is the use of biocorrodible metal alloys because, typically, a permanent support function by the stent is not necessary; the initially damaged vessel can regenerate. Thus, it is suggested in DE 197 31 021 A1 that medical implants be molded from a metallic material the main component of which is an element from the group of alkali metals, alkaline earth metals, zinc and aluminum. Alloys based on magnesium and zinc are described as especially suitable. Secondary components of the alloys may be manganese, cobalt, nickel, chromium, copper, cadmium, lead, tin, thorium, zirconium, silver, gold, palladium, platinum, silicon, calcium, lithium, aluminum and zinc. Furthermore, the use of a biocorrodible magnesium alloy having a proportion of magnesium >90%, yttrium 3.7-5.5%, rare earth metals 1.5-4.4%, and the balance being <1% is known from DE 102 53 634 A1, which is suitable, in particular, for producing an endoprosthesis, e.g., in the form of a self-expanding or balloon-expandable stent.

Biocorrodible implants thus represent a promising approach to reducing the restenosis rate. One problem in the realization of systems of this type is the corrosion behavior of the implant. Thus a fragment formation by the corrosion process should be prevented if possible until the implant has grown into the vascular wall. Furthermore, the support function should be maintained over the period of the therapeutic target. The structural requirements cited above do not allow a free adaptation of the stent design with respect to its corrosion behavior—compromises have to be made.

### BRIEF SUMMARY OF THE INVENTION

The object of the present invention is to overcome the described disadvantages of the prior art. In particular, a stent is to be provided with an improved corrosion behavior. The object is attained with the provision of a stent with a structure of a biocorrodible magnesium alloy or tungsten alloy, which comprises a multiplicity of web sections connected to one another, wherein (i) the structure has a support structure of a number of first web sections connected to one another, which are designed to perform a function supporting the vascular wall or preserving the mechanical integrity of the stent after an expansion of the stent for a predetermined period of time and (ii) at least one second web section is present, which a) is directly connected to a selected first web section, b) does not perform a function supporting the vascular wall or preserving the mechanical integrity of the stent after the expansion of the stent for the predetermined period of time, and c) has a smaller average grain size than the first web sections of the support structure from (i).

### DETAILED DESCRIPTION OF THE INVENTION

The invention is based on the finding that a smaller average grain size in a metallic material leads to an increased corrosion rate in this area. The so-called grain refinement lies in that a smaller, finer grain is achieved in the structure of the metallic material, e.g., through local heat treatment. In a body which can comprise. e.g., a single metallic material, areas with different average grain size and thus with different corrosion properties can thus be produced.

According to the invention it is now provided to use these differences in the corrosion behavior of the same metallic material depending on its grain size to influence the corrosion behavior of a stent with a structure of a biocorrodible magnesium alloy or tungsten alloy in a first phase of the degradation. The cited period of time begins immediately after the implantation and ends at a predetermined time that corresponds to the therapeutic stipulations and requirements for safety. Preferably this period extends over 2 to 6 weeks immediately after the implantation. As a rule within this period the stent has grown into the vascular wall and the vascular wall is regenerated to the extent that a further support function is no longer necessary.

A bearing structure for the purposes of the invention combines the web sections that perform a support function for the vascular wall over the predetermined period and the function supporting the construction (i.e., the function preserving the mechanical integrity of the implant) over the predetermined period. These are in particular the web sections without which the support function and bearing function no longer meets the requirements at the implantation site.

Web sections with reduced grain size are now provided in certain areas of the bearing structure through a targeted heat treatment. These special web sections are created such that their average grain size is smaller than the average grain size of the respective web section of the bearing structure to which they are connected. Through the production of smaller average grain sizes more grain boundaries per mm² or more grain boundary surfaces per mm³ metallic material are provided which can represent the points of attack for a corrosive action. The web section with smaller average grain size therefore has an increased corrosion rate, thus is corroded first and thus forms a defined nucleation point for the start of the corrosion of the stent according to the invention. Through a suitable selection of the areas with smaller grain size, it can thus be established in a controlled manner where in the stent the corrosion should start and from where it should spread over the course of time. The web section of the bearing structure connected to the web section with smaller grain size is thereby temporarily stabilized. A duration of the stabilizing effect can be influenced via the selection of the number, extension and the arrangement of the web sections with smaller grain size in the stent, namely with the proviso that at least the predetermined period cited above is ensured. Of course, the web section with smaller average grain size itself does not have to perform a bearing function or serve to maintain the mechanical integrity of the implant. A special advantage of the concept according to the invention lies in that the same biodegradable metallic material is used for the entire implant and a fine adjustment of the material properties with respect to the corrosion behavior is achieved through a different heat treatment of the material in certain web sections. Suitable differences in the average grain size between the first web sections and the at least one second web section are easily produced for one skilled in the art, with the aid of only routine tests and the known Hall-Petch relation.

On the one hand, it is desirable with stents to achieve a high bending flexibility early, but on the other hand to obtain the longest possible radial strength or integrity. The connector structures that connect the stent elements with radial support function to one another are 1s thereby necessary only for the stent assembly and application. After the implantation and stent integration into the vascular wall, only the ring elements are then required which take over the bearing function for holding open the vessel. The stents according to the invention are particularly suitable for applications of this type when, e.g., one or more of the connectors are embodied as second web sections with smaller average grain size.

On the other hand, with growing vessels (e.g., stents for pediatric applications) it is desirable not to impede their growth with ring elements of a stent. Defined corrosion points or degradation points in the ring element of the stent here guarantee a disintegration of the stent, which maintains a still adequate support function for the remaining stent elements without essentially impeding the radial growth of the vessel. The stents according to the invention are particularly suitable for this, since selected “predetermined breaking points” in the ring element of the stent can be established and produced in a targeted manner without having to resort to producing the stent from different materials.

The production of a smaller average grain size in the second web section can be achieved in different ways. According to the invention, the finer grain structure of the at least one second web section can be produced by local heat treatment. Fundamentally, any method of local heat treatment is suitable, provided that it permits a treatment of the selected second web sections at a temperature that is higher than the temperature to which the first web sections are exposed. One skilled in the art is familiar with suitable forms of local heat treatment or he will have no difficulty adapting known methods for local heat treatment within the scope of routine tests such that these methods can be applied to stents and lead to the stents according to the invention. Preferably the local heat treatment is selected, e.g., from a treatment of the stent with a laser beam or with an electron beam.

In a preferred embodiment of the stent according to the invention, previously defined sections of the at least one second web section are fed to the local heat treatment in a targeted manner.

The production of a smaller average grain size in at least one second web section can be carried out at each stage of the stent production and stent provision up to the insertion of the stent. The finally prepared, crimped stent can thus be subjected to a local heat treatment as can stent precursor products. The stent can thereby be cut from a tube. Subsequently areas are selected in a targeted manner for the local heat treatment and subjected to a corresponding treatment. Alternatively, for example, the semi-finished product, thus, e.g., the tube not yet finally cut to size, can also be subjected to a local heat treatment at those locations that are later to represent a second web section for the purposes of the invention.

The biocorrodible metallic material is preferably a biocorrodible magnesium alloy or tungsten alloy; in particular the biocorrodible metallic material is a magnesium alloy. In this case alloy means a metallic structure, the main component of which is magnesium or tungsten. The main component is the alloy component, the weight proportion of which in the alloy is highest. A proportion of the main component is preferably more than 50% by weight, in particular more than 70% by weight. Preferably, the alloy of the stent according to the invention does not essentially contain any iron.

If the material is a magnesium alloy, this preferably contains yttrium and other rare earth metals, since an alloy of this type is distinguished by its physicochemical properties and high biocompatibility, in particular also its degradation products.

Particularly preferably a magnesium alloy is used with the composition rare earth metals 5.2-9.9% by weight, thereof yttrium 3.7-5.5% by weight, and the balance being <1% by weight, wherein magnesium accounts for the proportion of the alloy to make up 100% by weight. This magnesium alloy has already confirmed its particular suitability in experiments and in initial clinical trials, i.e., the magnesium alloy exhibits a high biocompatibility, favorable processing properties, good mechanical characteristics and a corrosion behavior adequate for the purposes of use. The collective term “rare earth metals” in this case means scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) and lutetium (71).

The alloys of the elements magnesium or tungsten are to be selected in their composition such that they are biocorrodible. For the purposes of the invention alloys are referred to as biocorrodible with which a degradation occurs in a physiological environment which ultimately results in the entire implant or the part of the implant made of the material losing its mechanical integrity. Artificial plasma is used as a test medium for testing the corrosion behavior of a possible alloy, as is stipulated for biocorrosion assays according to EN ISO 10993-15:2000 (composition NaCl 6.8 g/l. CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l). For this purpose, a sample of the alloy to be tested is stored in a closed sample container with a defined quantity of the test medium at 37° C. At time intervals, coordinated with the corrosion behavior to be expected, of a few hours up to several months, the samples are removed and tested for corrosion traces in the known manner. The artificial plasma according to EN ISO 10993-15:2000 corresponds to a medium similar to blood and thus represents a possibility for simulating a physiological environment for the purposes of the invention in a reproducible manner.

In this case the term corrosion refers to the reaction of the metallic material with its environment, wherein a measurable change of the material is caused which, when the material is used in a component, leads to an impairment of the function of the component. A corrosion system comprises the corroding metallic material and a liquid corrosion medium, which simulates the conditions in a physiological environment in its composition, or is a physiological medium, in particular, blood. In terms of material, the corrosion influences factors such as the composition and pretreatment of the alloy, microscopic and submicroscopic inhomogeneities, edge zone properties, temperature state and state of stress and, in particular, the composition of a layer covering the surface. On the side of the medium, the corrosion process is influenced by conductivity, temperature, temperature gradients, acidity, volume-surface ratio, concentration difference and flow velocity.

In a further aspect, the invention relates to a method for producing a stent according to the invention, comprising the steps
a) Provision of a stent or of a stent precursor product with at least one established first and second web section,
b) Local treatment of the established at least one second web section with heat at a temperature that is higher than the temperature to which the at least one web section is exposed.

In this method stents or stent precursor products with a structure of a biocorrodible magnesium alloy or tungsten alloy are used.

The method according to the invention can be carried out at any stage in the stent production and stent provision up to the insertion of the stent. Therefore the finally prepared, crimped stent can he subjected to a local heat treatment just like stent precursor products. The stent can thereby be cut from a tube. Subsequently, areas are selected in a targeted manner for the local heat treatment and subjected to a corresponding treatment. Alternatively. e.g., the semi-finished product, thus, e.g., the tube not yet finally cut to size, can also be subjected to a local heat treatment at those locations which are later to represent a second web section for the purposes of the invention.

Fundamentally, any method for local heat treatment is suitable, provided that it permits a local treatment of the established at least one second web section with heat at a temperature that is higher than the temperature to which the at least one first web section is exposed. One skilled in the art is familiar with suitable forms of local heat treatment or he will have no difficulty in adapting known methods for local heat treatment within the scope of routine tests such that they can be applied to stents. Preferably the local heat treatment is selected. e.g., from a treatment of the stent with a laser beam or an electron beam.

What is claimed is:

1. A stent with a structure of a biocorrodible magnesium alloy or tungsten alloy, which comprises a multiplicity of web sections connected to one another, wherein
(i) The structure has a support structure of a number of first web sections connected to one another, which are designed to perform a function supporting the vascular wall or preserving the mechanical integrity of the stent after an expansion of the stent for a predetermined period of time; and
(ii) At least one second web section is present, which
a) Is directly connected to a selected first web section,
b) Does not perform a function supporting the vascular wall or preserving the mechanical integrity of the stent after the expansion of the stent for the predetermined period of time, and
c) Has a smaller average grain size than the first web sections of the support structure from (i).

2. The stent according to claim 1, characterized in that the smaller average grain size of the at least one second web section can be produced by local heat treatment.

3. The stent according to claim 2, characterized in that the local heat treatment is selected from the treatment of the stent with a laser beam or with an electron beam.

4. The stent according to claim 1, characterized in that previously defined sections of the at least one second web section are fed to the local heat treatment in a targeted manner.

5. A method for producing the stent according to claim 1, comprising the following steps:
a) Providing a stent or a stent precursor product with at least one established first and second web section, and
b) Locally treating the established at least one second web section with heat at a temperature that is higher than the temperature to which the at least one web section is exposed.

6. The method according to claim 5, wherein a laser beam is used in step b).

7. The method according to claim 5, wherein an electron beam is used in step b).
